(19) United States
(12) Patent Application Publication
(10) Pub. No.: US 2009/0022850 A1
(43) Pub. Date: Jan. 22, 2009

(54) ACID TOLERANT LEUCONOSTOC MESENTEROIDES WITH EXCELLENT MANNITOL PRODUCTIVITY AND METHOD FOR PRODUCING A KIMCHI USING THE SAME

(75) Inventors: Ji-Young Oh, Gyeonggi-do (KR); Si-Young Yang, Gyeonggi-do (KR); Min-Soo Kwon, Gyeonggi-do (KR); Jiyoung Oh, Gyeonggi-do (KR); Jin-Hyuk Lee, Gyeonggi-do (KR); Gyeong-Ho Han, Gyeonggi-do (KR); Ui-Chan Koh, Gyeonggi-do (KR)

Correspondence Address: BUCHANAN, INGERSOLL & ROONEY PC, POST OFFICE BOX 1404, ALEXANDRIA, VA, 22313-1404, US

(73) Assignee: DAESANG FNF CORPORATION, Seoul (KR)

(21) Appl. No.: 12/087,161

(22) PCT Filed: Dec. 29, 2005
(86) PCT No.: PCT/KR2005/004635

§ 371 Date: Jun. 27, 2008

(51) Int. Cl. A23B 7/10; C12N 1/20

(52) U.S. Cl. 426/61; 435/252.9

(57) ABSTRACT

The present invention relates to acid tolerant Leuconostoc mesenteroides with excellent mannitol productivity and the use thereof. The inventive lactic acid bacteria is useful as a food additive composition and a Kimchi starter. Kimchi prepared with the addition of the inventive lactic acid bacteria is maintained at a suitable ripening degree over an extended period of time than to the prior Kimchis, and has excellent sensory properties, including excellent refreshing taste, weak sour odor and a very soft quality of sour taste.

### FIELD OF THE INVENTION

The present invention relates to acid-tolerant lactic acid bacteria with excellent mannitol productivity and the use thereof.

### BACKGROUND OF THE INVENTION

Kimchi is Korean traditional fermented food, which is prepared by adding red pepper powder, garlic, ginger, Welsh onion, radish and the like to salted Chinese cabbage and fermenting the mixture at low temperatures in order to ensure the preservation and ripening of the product. Up to the 1990's, Kimchi has been produced mainly at home, but with rapid economic growth, an increase in the national income, changes in industrial structures and living environment, it began to be produced on a commercial scale after the 1990's. In the case of Kimchi produced on a commercial scale, it was very difficult to maintain the quality or taste of the commercial Kimchi at a constant level in the desired condition, since naturally occurring strains that induce the fermentation of Kimchi and produce the characteristic taste of Kimchi vary depending on the origin of raw materials, seasons, and fermentation conditions. Thus, there has been a need for the development of commercial Kimchi, the quality and taste of which are consistent and excellent.

The clean and refreshing taste of Kimchi is caused mainly because Leuconostoc mesenteroides, which are heterofermentative lactic acid bacteria of Kimchi, increase during the ripening of Kimchi (So M. H. and Kim Y. B., Korean J. Food Sci. Technol., 27(4):495-505, 1995; So M. H. and Kim Y. B., Korean J Food Sci. Technol., 27(4):506-515, 1995). The Leuconostoc mesenteroides starts to grow in the initial stage of production of Kimchi and produces metabolites, such as carbon dioxide, lactic acid, acetic acid, ethanol and mannitol, thus fermenting Kimchi having a complex and unique taste. Also, this strain causes the produced carbon dioxide to maintain the inside of Kimchi in anaerobic conditions thereby strongly inhibit the propagation of aerobic bacteria, thus maintaining normal fermentation of Kimchi. However, when Kimchi is reached the middle stage of fermentation, the number of Leuconostoc mesenteroides bacteria will rapidly decrease, and Lactobacillus plantarium, which is a homofermentative lactic acid bacteria strain, will be actively proliferated. The Lactobacillus plantarium is a main cause for the sour taste of Kimchi and is known to be involved in acidification. The Lactobacillus plantarium produces large amounts of lactic acid and can be grown even in an acidic environment of pH 3.0. Thus, in the last stage of fermentation of Kimchi, the growth of Leuconostoc mesenteroides, a lactic acid bacteria relatively weak to acid, is reduced while the growth of Lactobacillus plantarium that causes the sour taste of Kimchi is increased. Therefore, the refreshing and rich taste of Kimchi will disappear. Accordingly, maintaining the growth of Leuconostoc mesenteroides in Kimchi during a storage period to MAINTAIN the refreshing and clean taste of Kimchi is critical to produce Kimchi having good quality and taste.

Furthermore, mannitol which is produced by Kimchi fermentation is produced from fructose derived from the Kimchi by mannitoldehydrogenase after the optimum Kimchi-ripening stage give and Kimchi a refreshing and soft sweet taste, while it has the effects of inhibiting the over-ripening of Kimchi and reducing the sour odor and taste of Kimchi (Hawer W. D., Ahn H. S. Report of Korea Food Research Institute, S9-3; Kang S. C., Yun J. W., Ro T. W., Korean J. Biotechnol. Bioeng., 11(2), 1996).

Accordingly, many studies to control the taste and fermentation of Kimchi using lactic acid bacteria isolated from Kimchi as a starter in the production of Kimchi have recently been conducted. Particularly, studies to improve the quality of Kimchi using Leuconostoc mesenteroides have been conducted. Korean Patent Registration No. 1989-4894 discloses a method for producing Kimchi inhibited acidification and extended preservation by adding sodium hypochlorite and Leuconostoc mesenteroides. Also, Korean Patent Registration No. 0181009 discloses a method for producing Kimchi having good tastes and also delayed acidification by inhibiting growth of a Lactobacillus sp. strain, wherein the method comprises adding a mixture of Leuconostoc paramesenteroides and Leuconostoc mesenteroides. However, the Leuconostoc mesenteroides have problems in that, they decrease in number with gradual progression of Kimchi fermentation, and thus cannot continuously maintain the taste of Kimchi.

In an attempt to overcome these problems, Korean Patent Publication No. 1996-1940 discloses that an acid-tolerant variant strain was prepared by treating the Leuconostoc sp. strain with ultraviolet rays in order to improve the acid tolerance of the Leuconostoc sp., and the addition of the variant strain as a starter in the production of Kimchi resulted in improved sensory properties of Kimchi and delayed acidification of Kimchi. The Kimchi produced by adding said acid-tolerant variant strain had good tastes and showed delayed acidification, but did not show the inhibition of potent acid tolerant strain Lactobacillus plantarium which causes the sour taste of Kimchi.

Accordingly, there is an urgent need for the development of a lactic acid bacteria starter which allows the refreshing and clean taste of Kimchi to be maintained for a long period of time.

### DETAILED DESCRIPTION OF THE INVENTION

### Technical Problem

It is an object of the present invention to provide an acid-tolerant lactic acid bacteria from Kimchi with excellent mannitol productivity and the use thereof.

### Technical Solution

To achieve the above object, in one aspect, the present invention provides Leuconostoc mesenteroides DRC0512 (accession No. KCTC 10882BP) which has acid tolerance at a pH range of 3.5 to 5.0 and excellent mannitol productivity.

In another aspect, the present invention provides a food additive composition comprising said strain or its culture broth.

In still another aspect, the present invention provides a method for preparing Kimchi, comprising adding said strain or its culture broth. Hereinafter, the present invention will be described in detail.

The present invention is characterized in that it provides acid tolerant Leuconostoc mesenteroides with excellent mannitol productivity.

In the present invention, an acid tolerant lactic acid bacteria, was isolated from Kimchi having the best taste. The isolated lactic acid bacteria strain was naturally improved in a medium comprising fructose to obtain a novel strain having excellent mannitol productivity along with acid tolerance. The results of identification of the obtained strain showed that the strain belongs to Leuconostoc mesenteroides. The obtained strain was named “Leuconostoc mesenteroides DRC0512”, and was deposited under accession No. KCTC 10882BP on Dec. 14, 2005 with the Korean Collection for Type Cultures (KCTC), Korean Research Institute of Bioscience and Biotechnology (52, Oun-dong, Yusong-ku, Taejon, Korea), which is an International Depository Authority under the Budapest Treaty. The deposit shall be maintained in viable condition at the KCTC during the entire term of the issued patent and shall be made available to any person or entity for non-commercial use without restriction, but in accordance with the provisions of the law governing the deposit.

Leuconostoc mesenteroides DRC0512 of the present invention has acid tolerance at a pH range of 3.5 to 5.0. The proliferation of general Leuconostoc mesenteroides strains is rapidly reduced at acidic conditions, particularly pH of less than 4.0, whereas the lactic acid bacteria of the present invention actively grew even at an acidic condition of pH 3.5, indicating high degree of acid tolerance (see Table 3).

Also, Leuconostoc mesenteroides DRC0512 of the present invention has excellent mannitol productivity (see Table 1). In particular, Leuconostoc mesenteroides DRC0512 of the present invention has about 30% higher mannitol productivity in a sugar comprising medium than the prior Leuconostoc pseusomesenteroides ATCC12291 known to have excellent mannitol productivity (N. V. Weymarn, M. Hujanen, M. Leisola, Process Biochemistry, 37, 1207-1213, 2002) (see FIG. 1).

Leuconostoc mesenteroides DRC0512 of the present invention, or a culture broth thereof, can be used as a food additive composition, such as Kimchi, drinks, baby food and the like. In addition to Leuconostoc mesenteroides DRC0512 of the present invention or its culture broth, the food additive composition of the present invention may additionally contain components used in general food additive compositions, such as a carrier, an excipient, a preservative, and spices, when needed. Moreover, Leuconostoc mesenteroides DRC0512 of the present invention can be used as a starter for the preparation of fermented products. The fermented products include fermented uncooked food products, cheese, Kimchi and the like. Fermented products comprising Leuconostoc mesenteroides DRC0512 of the present invention or its culture broth can be prepared by a conventional method known in the art. For example, fermented uncooked food products can be produced by treating cereal powder, such as unpolished rice and unshelled grains of adlay, with Leuconostoc mesenteroides DRC0512 of the present invention or a mixture of two or three lactic acid, including the inventive strain, fermenting the treated cereal powder at a suitable temperature, and adding various agricultural products, such as white soybean, glutinous rice and kaoliang so as to provide excellent nutritional balance and preference. Particularly, Leuconostoc mesenteroides DRC0512 of the present invention, or its culture broth, can be added to prepare Kimchi. Preferably, Kimchi can be prepared by adding general dressing materials, such as red pepper powder, garlic, ginger, Welsh onion, radish shreds and sugar, to salted Chinese cabbage, and then adding Leuconostoc mesenteroides DRC0512 of the present invention or its culture broth to the mixture.

Kimchi prepared with the addition of the inventive lactic acid bacteria had a slow rate of increase in acidity (see FIG. 2). Also, the prepared Kimchi had weak sour odor and weak sour taste after ripening, because of a high content of mannitol in the Kimchi, and was evaluated to be excellent in general sensory properties (see FIG. 3 and Table 5). Particularly, if Leuconostoc mesenteroides DRC0512 of the present invention is added to Kimchi, it will be present as a dominant species in the Kimchi and take leading role in the Kimchi fermentation. Thus, Kimchi prepared with the addition of Leuconostoc mesenteroides DRC0512 of the present invention has a slow rate of increase in acidity, and so can be maintained at a suitable ripening degree (acidity of 0.4-0.8%) for a long period of time. Also, the prepared Kimchi can have a clean and refreshing taste because of a high content of mannitol, and can preserve the taste for a long time by suppressing sour taste.

In the present invention, the strain Leuconostoc mesenteroides DRC0512 can be used in the form of a cell wall fraction resulting from the disruption thereof, viable bacteria, nonviable bacteria, and dry bacteria. Also, in the present invention, the culture broth of Leuconostoc mesenteroides DRC0512 include a culture broth itself resulting from culturing in a liquid medium, and a filtrate (centrifuged supernatant) obtained by filtering or centrifuging the culture broth to remove strain. Furthermore, the culture broth is also includes one obtained by drying (e.g., freeze drying) the culture broth, and then powdering.

If the inventive lactic acid bacteria strain is used to produce Kimchi, the bacteria strain itself may be added directly to Kimchi. Preferably a culture broth of the bacterial strain may be added. The inventive culture broth of Leuconostoc mesenteroides DRC0512 used in the production of Kimchi is prepared primarily pre-culturing the strain in MRS broth (Difco.; comprising 10 g bacto peptone, 10 g beef extract, 5 g yeast extract, 20 g glucose, 1 g Tween80, 2 g ammonium citrate, 2 g dipotassium phosphate, 5 g sodium acetate, 0.1 g manganese sulfate, 0.05 g magnesium sulfate 0.05 g and 1 liter of deionized water); inoculating the primarily precultured culture broth into sterilized Chinese cabbage juice at a concentration of 0.5-1.0% (v/v) and then secondarily pre-culturing the inoculated preculture broth at a temperature of 25-30° C. for 18-24 hours; and inoculating the secondarily precultured culture broth into sterilized Chinese cabbage juice at a concentration of 0.5-1.0% (v/v) and then culturing the inoculated preculture broth at a temperature of 25-30° C. for 18-24 hours. Also, the cabbage juice is preferably prepared in the following manner: Salted Chinese cabbage was crushed and juiced, and the salinity of the juice is adjusted to 2.0-3.0%, preferably 2.5%, for the effective growth of the bacteria strain. Then, glucose is added to the cabbage juice at concentrations of 1.0-3.0% (w/v), and preferably 1.0% (w/v), followed by sterilization. The above-prepared culture broth of Leuconostoc mesenteroides DRC0512 is preferably added to Kimchi in an amount of 0.5-3.0% by weight of the Kimchi. If the culture broth of Leuconostoc mesenteroides DRC0512 is added in an amount of less than 0.5% by weight of Kimchi, an effect caused by the strain will be insignificant, and if it is added in an amount of more than 3.0% by weight, it will reduce the flavor of Kimchi and result in the over-ripening of Kimchi. Due to these problems, the amount of addition of the culture broth is preferably in the specified range. Most preferably, it may be added in an amount of 1.0% by weight.

Leuconostoc mesenteroides DRC0512 of the present invention can be cultured in large amounts according to a conventional method for culturing Leuconostoc sp. microorganisms. As a medium for culture, a medium comprising a carbon source, a nitrogen source, vitamins and minerals may be used, and for example, MRS (Man-Rogosa-Sharp) medium or Kimchi medium may be used. The Kimchi medium may be obtained by crushing and juicing ripened Kimchi, preferably ripened Kimchi at 4° C. for 24 hours after the preparation, and then sterilizing the crushed and juiced Kimchi. The culture of the microorganism can be performed in conventional conditions for the culture of Leuconostoc sp. microorganisms, for example, may be performed at 20-40° C. for about 10-40 hours. More preferably, it can be performed at 37° C. for about 18 hours. In order to remove the medium from the culture broth and recover only the concentrated bacterial cells, the culture broth can be subjected to a centrifugation or filtration process, if a person skilled in the art requires the process. The concentrated bacterial cells can be frozen or lyophilized according to a conventional method so as to preserve their activity.

### BRIEF DESCRIPTION OF THE DRAWINGS

FIG. 1 is the results of comparison of mannitol productions in sugar-comprising media between the inventive strain Leuconostoc mesenteroides DRC0512 and the prior lactic acid bacteria strains.

DRC0512: Leuconostoc mesenteroides DRC0512 according to the present invention;

ATCC12291: Leuconostoc pseudomesenteroides ATCC12291;

CH-3: Leuconostoc mesenteroides CH-3; and

OH-20: Leuconostoc mesenteroides OH-20.

FIG. 2 is graphic diagram showing the comparison of changes in acidity with the passage of ripening period between Kimchi produced with the addition of the inventive strain Leuconostoc mesenteroides DRC0512, Kimchis produced with the addition of lactic acid bacteria known in the prior art, and Kimchi produced without the addition of lactic acid bacteria.

—♦—Kimchi produced with the addition of the inventive strain Leuconostoc mesenteroides DRC0512;

—▪—: Kimchi produced with the addition of Leuconostoc pseudomesenteroides ATCC12291;

—-—: Kimchi produced with the addition of Leuconostoc mesenteroides CH-3;

——: Kimchi produced with the addition of Leuconostoc mesenteroides OH-20; and

—▴—: Kimchi produced without the addition of lactic acid bacteria.

FIG. 3 is a graphic diagram showing the comparison of changes in mannitol contents between Kimchi produced with the addition of the inventive Leuconostoc mesenteroides DRC0512, Kimchis produced with the addition of lactic acid bacteria known in the prior art, and Kimchi produced without the addition of lactic acid bacteria.

—♦—: Kimchi produced with the addition of the inventive strain Leuconostoc mesenteroides DRC0512;

—▪—: Kimchi produced with the addition of Leuconostoc pseudomesenteroides ATCC12291;

—-—: Kimchi produced with the addition of Leuconostoc mesenteroides CH-3;

——: Kimchi produced with the addition of Leuconostoc mesenteroides OH-20; and

—▴—: Kimchi produced without the addition of lactic acid bacteria.

### BEST MODE FOR CARRYING OUT THE INVENTION

Hereinafter, the present invention will be described in detail with reference to the flowing examples. However, the examples are given for illustrative purpose only and are not constructed to limit the scope of the present invention.

### EXAMPLE 1

### Preparation of Lactic Acid Bacteria with Acid Tolerance and Excellent Mannitol Productivity

<1-1> Preparation of Kimchi Sample for Isolation of Lactic Acid Bacteria, and Isolation of Lactic Acid Bacteria Therefrom

Sensory evaluation was performed on various Kimchis produced by a conventional production method of Kimchi, and Kimchi evaluated to be the best in taste was selected. The Kimchi was subjected to sensory evaluation with ripening at a low temperature of −1° C., and a Kimchi sample collected at a time point showing the best flavor was used as a Kimchi sample for the isolation of lactic acid bacteria. The Kimchi sample was diluted 10-fold with 0.85% saline solution, and 0.1 ml of the diluted sample was inoculated into each well of a PES (phenylethyl alcohol sucrose) agar medium plate (comprising 5 g tryptone, 0.5 g yeast extract, 20 g sucrose, 2 g ammonium sulfate, 1 g dipotassium phosphate, 0.244 g magnesium sulfate, 2.5 ml phenylethyl alcohol and 15 g agar in 1 liter of D.W) and spread with a glass rod. Then, the plate was incubated in a constant-temperature incubator at 25° C. for one day. Each of the produced colonies was streak-inoculated onto a PES agar plate and cultured at 25° C. for one day to isolate lactic acid bacteria colonies.

<1-2> Selection of Acid Tolerant Lactic Acid Bacteria

Each of the lactic acid colonies isolated in Example <1-1> was inoculated into 5 ml of each of MRS broths (Difco.; comprising 10 g bacto peptone, 10 g beef extract, 5 g yeast extract, 20 g glucose, 1 g Tween80, 2 g ammonium citrate, 2 g dipotassium phosphate, 5 g sodium acetate, 0.1 g manganese sulfate and 0.05 g magnesium sulfate in 1 liter of D.W) which have been adjusted to pH 3.0, 3.5, 4.0 and 5.0, respectively, with lactic acid. The growth or non-growth of the colonies was observed with culturing at 25° C. for 72 hours, and an acid tolerant bacteria strain having the most active growth even at the above a pH range was selected. The selected acid tolerant lactic acid bacteria strain was actively grown at a pH range of 3.5 to 5.0, indicating strong acid tolerance.

<1-3> Improvement into Bacterial Strain with Excellent Mannitol Productivity

In order to improve the lactic acid bacteria selected in Example <1-2> into excellent mannitol productivity strain the selected lactic acid bacteria strain was subcultured two times in 5 ml of MRS broth. Then, the lactic acid bacteria strain was inoculated into 5 ml of each of nutrient broths (comprising 5 g peptone, 2.5 g yeast extract and 10 g-90 g fructose in 1 liter of deionized water) supplemented with 1.0-9.0% (w/v) of fructose, at an inoculation concentration of 1.0% (v/v), and was then cultured at 25° C. for 24 hours. Then, the amount of the produced mannitol in each of the culture broths was measured, and strains in the first to fifth positions in order of the production amount of mannitol were selected. The selected five strains were inoculated into broths comprising 1.0-9.0% (w/v) of fructose, and the production amount of mannitol in each of the culture broths was again measured. The measurement of the mannitol production amount was carried out in the following manner. Each of the culture broths was centrifuged at 13,000 rpm for 3 minutes and then filtered through a 0.22 μm syringe filter. 20 μl of the filtered sample was analyzed by HPLC (high performance liquid chromatography) using an ultrapure H₂O (0.5 ml/min) solvent at 80° C.

Among the above strains, strains in the first to third positions in order of the production amount of mannitol were selected. The selected strains were further inoculated into broths comprising 1.0-9.0% (w/v) of fructose and measured for the production of mannitol. Among the broths, the broth comprising 6.0% (w/v) of fructose maintained mannitol production at the highest level. Thus, the strains were subcultured 10 times in the broth comprising 6.0% (w/v) of fructose, and among them, one strain with excellent mannitol productivity was finally selected (see Table 1).

TABLE 1

| | Patent strain | | | |
|---|---|---|---|---|
| | selected in | Improved | Improved | Improved |
| | Example <1-2> | strain 1 | strain 2 | strain 3 |
| Mannitol | 4.0 | 4.3 | 5.2 | 4.6 |
| content (%) | | | | |

* Nutrient broth comprising 60 g/l (i.e., 6.0% (w/v)) of fructose

### EXAMPLE 2

### Identification of Lactic Acid Bacteria Isolated and Improved in Example 1

<2-1> Analysis by Bergy's Manual of Systematic Bacteriology

The lactic acid bacteria (improved strain 2) finally selected in Example 1 was isolated as single colonies and then examined for morphological and biochemical properties according to Bergy's Manual of systematic bacteriology, and subjected to Gram staining. As a result, it could be found that the isolated strain is a gram-positive strain and has the shape of bacillus.

<2-2> Analysis with API System

The isolated strain was identified with an API system (La Balme-les-Grottes, France). First, the colony was taken using sterilized platinum and then suspended in 2 ml of sterilized distilled water. The suspension was suspended in 5 ml of sterilized distilled water at the concentration of MccFaland Standard Solution No. 2 provided in the API 50CH kit (BioMerieux, France). The suspension was homogenized in a liquid medium in the API 50CH kit and inoculated into each of 50 tubes of the API 50CH kit in an amount of 200 μl. Each of the tubes was covered with mineral oil and incubated at 30° C. for 48 hours. The culture broth was analyzed with the API system to examine the fermentation patterns of 49 carbohydrates, and the results were inputted into an ATB identification computer system. As a result, the lactic acid bacteria strain isolated and improved in Example 1 was confirmed to be a strain belonging to Leuconostoc mesenteroides, which has carbohydrate fermentation patterns shown in Table 2.

TABLE 2
Analysis results for carbohydrate fermentation patterns
of lactic acid bacteria of the present invention

| | | | | | | | |
|---|---|---|---|---|---|---|---|
| Control group | − | D-mannose | + | Salicin | − | Gentiobiose | − |
| Glycerol | − | L-solbose | − | Cellobise | − | D-turanose | + |
| Erythritol | − | Ramnose | − | Maltose | + | D-rizose | − |
| D-arabinose | − | Dulsitol | − | Lactose | − | D-tagatose | − |
| L-arabinose | + | Inositol | − | Mellibiose | + | D-fucose | − |
| Ribose | + | Mannitol | − | Saccharose | + | L-fucose | − |
| D-xylose | + | Sorbitol | − | Trehalose | + | D-arabitol | − |
| L-xylose | − | D-mannoside | − | Inulin | − | L-arabitol | − |
| Adonitol | − | D-glucoside | + | Mellesitose | − | Gluconate | − |
| Xyloside | − | Glucosamine | + | D- raffinose | + | 2-ceto- | − |
| | | | | | | gluconate | |
| Galactose | + | Amigdalin | − | Amidone | − | 5-ceto- | − |
| | | | | | | gluconate | |
| D-glucose | + | Albutin | − | Glycogen | − | | |
| D-fructose | + | Esculin | + | Xylitol | − | | |

<2-3> Analysis of 16S rDNA Sequence

According to a conventional method known in the art, the 16S rDNA sequence of the above-selected lactic acid bacteria was analyzed. As a result, it could be found that the 16S rDNA sequence of the lactic acid bacteria was 99% identical to the 16S rDNA sequence of Leuconostoc mesenteroides (data not shown).

Accordingly, the present inventors named the lactic acid bacteria strain “Leuconostoc mesenteroides DRC0512” and deposited the strain with the Korean Collection for Type Cultures, Korean Research Institute of Bioscience and Biotechnology on Dec. 14, 2005(accession No: KCTC 10882BP).

### EXAMPLE 3

### Comparison of Acid Tolerance Between Inventive Lactic Acid Bacteria and Prior Lactic Acid Bacteria

The comparison of acid tolerance between inventive Leuconostoc mesenteroides DRC0512 and prior lactic acid bacteria was performed. As the prior lactic acid bacteria strains (control groups), a Leuconostoc mesenteroides strain (KCCM-11325) and a Leuconostoc sp. strain (KFCC-10774) were used.

Each of the lactic acid bacteria strains was inoculated into 10 ml of each of MRS broths adjusted to pH 3.2, 3.5, 4.0 and 5.0, respectively with lactic acid. The inoculated bacteria strains were incubated at 25° C. for 72 hours while the observation of their growth or non-growth was performed. The results are shown in Table 3 below.

TABLE 3
Comparison results for acid tolerance

| | Inventive Leuconostoc | Leuconostoc | Leuconostoc |
|---|---|---|---|
| | mesenteroides | mesenteroides strain | sp. strain |
| PH | DRC0512 | (KCCM-11325) | (KFCC-10774) |
| 5.0 | + | + | + |
| 4.0 | + | + | + |
| 3.5 | + | − | − |
| 3.2 | − | − | − |

+: grown; and −: non-grown

As can be seen in Table 3 above, Leuconostoc mesenteroides DRC0512 of the present invention had an acid tolerance significantly stronger than that of the prior Leuconostoc sp. strains and maintained active growth at a pH range of 3.5 to 5.0.

### EXAMPLE 4

### Comparison of Mannitol Productivity Between Inventive Lactic Acid Bacteria and Prior Lactic Acid Bacteria

The comparison of mannitol productivity between the inventive Leuconostoc mesenteroides DRC0512 and the prior lactic acid bacteria was performed. As the prior lactic acid bacteria (control group), Leuconostoc pseudomesenteroides ATCC12291 known to have excellent mannitol productivity, and Leuconostoc mesenteroides CH-3 and OH-20 isolated from Kimchi, were used. Each of the lactic acid bacteria strains was inoculated into 5 ml of each of nutrient broths (comprising 5 g peptone, 2.5 g yeast extract and 10-90 g fructose in 1 liter of D.W) comprising 2% (w/v) of fructose, at an inoculation concentration of 1.0% (v/v), and then cultured at 10° C. for 5-10 days. Thereafter, the production amount of mannitol in each of the broths was measured in the same manner as described in Example <1-3>.

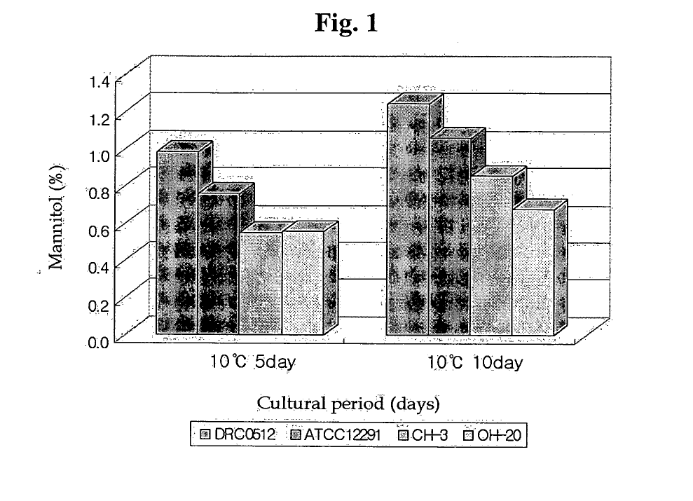

As a result, as shown in FIG. 1, the inventive lactic acid bacteria strain produced mannitol at significantly higher levels than those of the prior Leuconostoc mesenteroides strains (CH-3 and OH-20) isolated from Kimchi. Also, the inventive lactic acid bacteria strain produced mannitol at a higher level than that of the prior Leuconostoc pseudomesenteroides ATCC12291 known to excellent mannitol productivity.

### EXAMPLE 5

### Production of Kimchi using Leuconostoc mesenteroides DRC0512 and Analysis of Properties of Produced Kimchi

<5-1> Preparation of Culture Broth of Leuconostoc Mesenteroides DRC0512 for addition to Kimchi

First, the inventive Leuconostoc mesenteroides DRC0512 strain was inoculated into 5 ml of MRS broth and primarily pre-cultured at 25° C. for 24 hours. Then, the primarily pre-cultured culture broth was inoculated into Chinese cabbage juice at a concentration of 1.0% (v/v) and secondarily pre-cultured at 25° C. for 24 hours. In this regard, the Chinese cabbage juice was prepared in the following manner. Salted Chinese cabbage was crushed and juiced with a blender and adjusted to a salinity of 2.5% using boiled salt. Then, glucose was added thereto at a concentration of 1.0% (w/v), followed by sterilization, thereby preparing cabbage juice. Next, the secondarily pre-cultured culture broth was inoculated into the sterilized cabbage juice at a concentration of 1.0% and cultured at 25° C. for 18 hours.

<5-2> Preparation of Kimchi

First, Chinese cabbage was salted to a salinity of 2.3%. Kimchi dressings including red pepper powder(2.5 wt %), garlic(2 wt %), ginger(0.5 wt %), Welsh onion(2 wt %), radish shreds(9 wt %) and sugar(0.5 wt %) were added to To the salted cabbage(82.5 wt %). Then, the culture broth of the inventive Leuconostoc mesenteroides DRC0512, prepared in Example <5-1>, was added as a starter in an amount of 1.0% (w/w) based on the total weight of Kimchi. As positive control groups, Leuconostoc pseudomesenteroides ATCC12291 (positive control group 1) known excellent mannitol productivity, and Leuconostoc mesenteroides CH-3 (positive control group 2) and OH-3 (positive control group 3) isolated from Kimchi, were used. Also, as a negative control group, Kimchi produced in the same manner without adding a lactic acid bacteria was used. Thereafter, each of the produced Kimchis was stored at 10° C., the general ripening temperature of Kimchi, while they were measured for acidity, lactic acid bacteria number and mannitol production and subjected to sensory evaluation.

<5-3> Measurement of Acidity of Kimchi

The acidity of the each Kimchi produced in Example <5-2> was measured and compared to each other. For this purpose, 100 g of each Kimchi was ground and then filtered through gauze thereby preparing Kimchi juice. 20 ml of the Kimchi juice was neutralized with 0.1N NaOH to pH 8.1, and the consumed amount of NaOH was converted into lactic acid content (%) according to the following equation so as to determine acidity.

Acidity (%)={0.00908×F(0.1 N NaOH factor)×addition amount of 0.1N NaOH}/sample weight

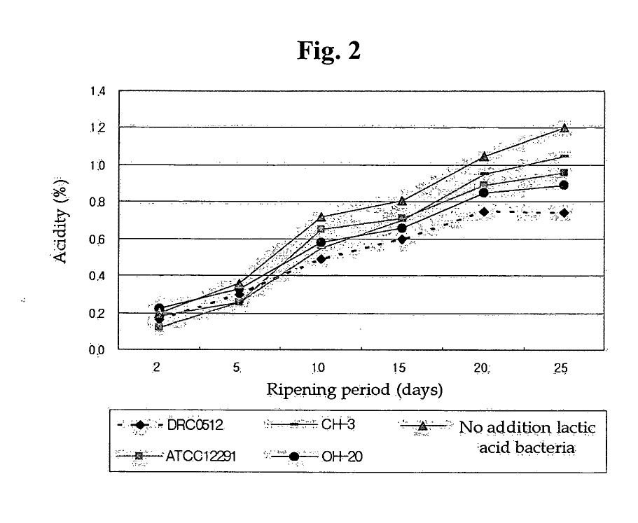

As a result, as shown in FIG. 2, the Kimchis had no great difference in acidity at the initial ripening stage, but the difference in acidity became wider as the ripening period became longer. The Kimchi produced using the inventive lactic acid bacteria showed a slow rate of increase in acidity as compared to those of the other Kimchis. Also, even 20 days after ripening of the Kimchi comprising the inventive lactic acid bacteria, the Kimchi maintained a suitable ripening degree (0.4-0.8% acidity). On the other hand, the Kimchis comprising the other lactic acid bacteria all showed an acidity exceeding 0.8% at 20 days of ripening and a further increase in acidity at 25 days of ripening. The Kimchi (positive control group 1) produced using prior Leuconostoc pseudomesenteroides ATCC12291 known to have excellent productivity had the lowest initial acidity, but showed a rapid increase in acidity after 5 days of ripening and deviated from a suitable ripening degree at 20 days of ripening.

<5-4> Measurement of Number of Lactic Acid Bacteria

The number of lactic acid bacteria in each of the Kimchis produced in Example <5-2> was measured. For this purpose, each of the Kimchis ripened at 10° C. for 10 days was diluted 10-fold with 0.85% saline solution to prepare Kimchi dilutions. Then, to measure the total number of lactic acid bacteria, 0.1 ml of each of the Kimchi dilutions was inoculated onto an MRS agar medium plate and spread with a glass rod. Also, to measure the number of Leuconostoc bacteria, 0.1 ml of each of the Kimchi dilutions was inoculated into a PES agar medium (comprising 5 g tryptone, 0.5 g yeast extract, 20 g sucrose, 2 g ammonium sulfate, 1 g dipotassium phosphate, 0.244 g magnesium sulfate, 2.5 ml phenylethyl alcohol and 15 g agar in 1 liter of deionized water) and spread with a glass rod. Each of the Kimchi dilution-spread agar plates was incubated in a constant-temperature incubator at 25° C. for 2 days. The number of the produced colonies was counted as the number of each of the bacteria. Also, dominant percentage (%) was calculated as the ratio of the number of starter lactic acid bacteria (bacteria having a morphological type coinciding with that of starter lactic acid bacteria in the Leuconostoc sp. strains appearing on the PES agar plate) relative to the total number of lactic acid bacteria (see the following equation). The results are shown in Table.

Dominant percentage (%)=(number of Leuconostoc sp. having morphological type coinciding with starter lactic acid/total number of lactic acid bacteria)×100

TABLE 4
Measurement results for number of lactic acid bacteria
in Kimchi produced using inventive lactic acid bacteria

| | | | Dominant |
|---|---|---|---|
| | Total number of | | percentage |
| | lactic acid | Leuconostoc sp. | (%) of lactic |
| Kind of Kimchi | bacteria | strain | acid bacteria |
| Kimchi | 9.86 × 10⁸ | 8.14 × 10⁸ | 72.5 |
| comprising | | | |
| inventive | | | |
| Leuconostoc | | | |
| mesenteroides | | | |
| DRC0512 | | | |
| Positive control | 7.21 × 10⁸ | 4.59 × 10⁸ | 42.6 |
| group 1 | | | |
| Positive control | 6.25 × 10⁸ | 3.29 × 10⁸ | 50.4 |
| group 2 | | | |
| Positive control | 5.22 × 10⁸ | 3.28 × 10⁸ | 43.7 |
| group 3 | | | |
| Negative control | 8.9 × 10⁸ | 7.32 × 10⁷ | — |
| group | | | |

* Results (number of lactic acid bacteria, cfu/g) of ripening at 10° C. for 10 days.

Dominant percentage: appearance percentage of starter lactic acid bacteria relative to total lactic acid bacteria; the negative control group was not calculated.

As a result, as shown in Table 4, the Kimchi prepared by adding the inventive lactic acid bacteria as a starter was significantly larger in the number of Leuconostoc sp. than those of the Kimchis (positive control groups 1 to 3) produced using the prior lactic acid bacteria. Also, the examination results for the dominant percentage showed that the dominant percentage of the inventive starter lactic acid bacteria in the Kimchi produced with the addition of the inventive lactic acid bacteria was 72.5%, the highest value. The Leuconostoc mesenteroides DRC0512 of the present invention has excellent growth resulting from strong acid tolerance and is also present as a dominant species in Kimchi and take leading role in the fermentation, so that it allows the fresh taste of Kimchi to be maintained over an extended period of time.

<5-5> Measurement of Mannitol Content

The mannitol contents of the Kimchis prepared in Example <5-2> were measured and compared to each other. Kimchi juice was prepared from each of the Kimchis in the same manner as described in Example <5-2> and then centrifuged at 13,000 rpm for 3 minutes, followed by filtration through a 0.22 μm syringe filter. 20 μl of the filtered sample was analyzed by HPLC (high performance liquid chromatography) using ultrapure H₂O (0.5 ml/min) solvent at 80° C.

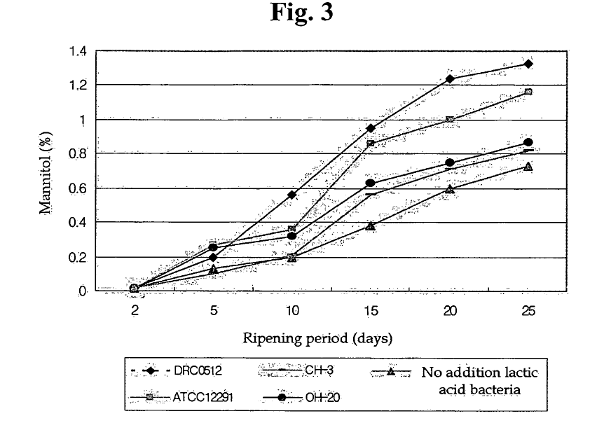

As a result, as shown in FIG. 3, the Kimchi produced using the inventive Leuconostoc mesenteroides DRC0512 had the highest mannitol content, which was shown to increase with the passage of ripening period. Also, the Kimchi produced using the inventive bacteria strain had a mannitol content higher than that of the Kimchi (positive control group 1) produced using the prior Leuconostoc pseodomesenteroidesATCC12291 known to have excellent mannitol productivity.

<5-6> Sensory Test

Each of the Kimchis (0.6-0.7% acidities) prepared in Example <5-2> was subjected to sensory test by 50 trained panels. The test was performed by a hedonic scaling method with 5 points as a perfect score, and the test of significance was carried out by t-test. The results are shown in Table.

TABLE 5

| | | | | | Quality | |
|---|---|---|---|---|---|---|
| | Overall | Carbonated | Refreshing | Clean | of sour | Sour |
| Kind of Kimchi | taste | taste | taste | taste | taste* | odor |
| Kimchi added | 4.3 | 4.1 | 3.7 | 3.9 | 2.1 | 2.5 |
| with inventive | | | | | | |
| lactic acid | | | | | | |
| bacteria | | | | | | |
| Positive control | 3.8 | 3.9 | 3.4 | 3.6 | 3.0 | 2.9 |
| group 1 | | | | | | |
| Positive control | 3.7 | 3.6 | 3.3 | 3.5 | 3.5 | 3.2 |
| group 2 | | | | | | |
| Positive control | 3.8 | 3.7 | 3.3 | 3.4 | 3.5 | 3.0 |
| group 3 | | | | | | |
| Negative control | 3.5 | 3.2 | 3.0 | 3.2 | 3.9 | 3.9 |
| group | | | | | | |

*The quality of sour taste: the intensity of sour taste, in which the lower the score, the more soft the sour taste.

As shown in Table 5, the Kimchi prepared using the inventive lactic acid bacteria was evaluated to have significantly excellent sensory properties as compared to the control group Kimchis. Particularly, the Kimchi produced according to the present invention was evaluated to have excellent refreshing taste, weak sour odor, and a very soft quality of sour taste.

### INDUSTRIAL APPLICABILITY

As described above, the lactic acid bacteria of the present invention has acid tolerance and excellent mannitol productivity. Thus, the inventive lactic acid bacteria is useful as a food additive composition and a Kimchi starter. The Kimchi prepared with the addition of the inventive lactic acid bacteria strain is maintained at a suitable ripening degree over an extended period of time as compared to prior Kimchis, and has excellent sensory properties, including excellent refreshing taste, weak sour odor and a very soft quality of sour taste.

1. A biologically pure culture of Leuconostoc mesenteroides having accession No: KCTC 10882BP.

2. A food additive composition, comprising the Leuconostoc mesenteroides DRC0512 of claim 1.

3. A method for preparing Kimchi, comprising adding the Leuconostoc mesenteroides DRC0512 of claim 1.

4. (canceled)
